(19) United States
(12) Plant Patent Application Publication
(10) Pub. No.: US 2014/0026275 P1
(43) Pub. Date: Jan. 23, 2014

(54) Microsorum plant namee 'Crested'

(50) ;

(71) Applicant: Jacob Bijl, Burgh-haamstede (NL)

(72) Inventor: Jacob Bijl, Burgh-haamstede (NL)

(21) Appl. No.: 13/507,689

(22) Filed: Jul. 20, 2012

(51) Int. Cl. A01H 9/00

(52) U.S. Cl. PLT/379

(57) ABSTRACT

A new and distinct Microsorum diversifolium cultivar named ‘Crested’ is disclosed, characterized by glossy, dark green crenated leaves, dark stipes and abushy growth form. The new variety is a Microsorum diversifolium, typically produced as an indoor ornamental plant.

### LATIN NAME OF THE GENUS AND SPECIES

Microsorum diversifolium

### VARIETY DENOMINATION

‘Crested’

### BACKGROUND OF THE INVENTION

The new cultivar is the product of chance discovery. The new variety originated as a naturally occurring mutation of an unpatented, unnamed proprietary variety of Microsorum diversifolium.

The new variety was discovered by the inventor, Jacob Johannes Bijl, a citizen of the Netherlands, during February 2010 in a commercial greenhouse belonging to the inventor in Burgh-Haamstede, the Netherlands. After identifying the new variety as a potentially interesting selection, the inventor continued confidential testing of ‘Crested’ to assess stability of the unique characteristics of this variety.

Asexual reproduction of the new cultivar ‘Crested’ was first performed at a commercial laboratory in Burgh-haamstede, the Netherlands, by tissue culture during Fall of 2010. Access to all plants was restricted, as plants were kept in a location that is not open to the public. Through subsequent propagation by tissue culture, multiple generations have been reproduced, which have shown that the unique features of this cultivar are stable and reproduced true to type.

### SUMMARY OF THE INVENTION

The cultivar ‘Crested’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Crested.’ These characteristics in combination distinguish ‘Crested’ as a new and distinct Microsorum diversifolium cultivar:
1. Bushy form.
2. Dark green glossy leaves with very dark stalks.

### Parent Comparison

Plants of the new cultivar ‘Crested’ are similar to the unnamed, unpatented parent, Microsorum diversifolium in most horticultural characteristics. The new variety, however, produces crenated leaves, whereas the unnamed unpatented parent does not.

### Commercial Comparison

Plants of the new cultivar ‘Crested’ are similar to the commercial variety Microsorum diversifolium ‘Kangaroo’, unpatented, in most horticultural characteristics. The new variety, however, produces darker stems, and forms a significantly bushier plant than ‘Kangaroo’.

### BRIEF DESCRIPTION OF THE PHOTOGRAPH

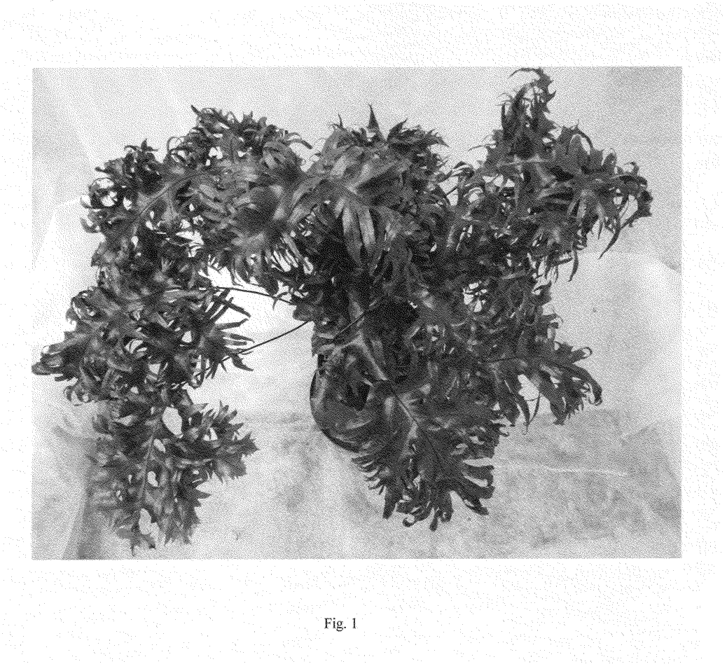

The accompanying photograph in FIG. 1 illustrates in full color a typical plant of ‘Crested’ grown in a commercial greenhouse in Burgh-Haamsede, The Netherlands. This plant is approximately 1 year old, shown planted in a 24 cm. container. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001, except where general terms of ordinary dictionary significance are used. The following observations and measurements describe ‘Crested’ plants grown in a greenhouse in Burgh-Haamsede, The Netherlands. This was done in June 2012 with a 1 year old in a 24 cm., 6 liter pot. Temperatures ranged from 18° C. to 22° C. at night to 18° C. to 30° C. during the day. No artificial light, photoperiodic treatments were given to the plants. No chemical treatments were given to plants. Measurements and numerical values represent averages of typical plant types.
Botanical classification: Microsorum diversifolium ‘Crested’
Propagation:
Time to initiate roots: Approximately 10 days from a rooted plantlet at approximately 22° C.
Time to produce rooted plantlet: Approximately 5 weeks at approximately 22° C.
Rhizome description:
   Color.—Green; near RHS 143A.
   Average diameter.—Approximately 0.7 cm.
   Other.—Rhizomes densely covered with bristly hairs, average length: 0.35 cm, colored brown; 200B and 200C.
Plant:
Growth habit: Spreading-creeping; leaves grow directly from rhizomes.
Plant shape: Flattened globular to flattened.
Height: Approximately 38.0 cm.
Plant spread: Approximately 97.5 cm.
Growth rate: Moderate to rapid.
Frond arrangement: Fronds grow directly from the base, irregular pattern.
Scales: No scales present
Stipe/stalk description:
   Length.—Approximately 26.9 cm.
   Width.—Approximately 0.3 cm.
   Color.—Brown; near RHS 200B.
   Strength.—Very strong.
   Texture.—Smooth, very glossy.
Foliage:
Leaf:
   Quantity per plant.—Average 60.
   Leaf type.—Single.
   Average leaf length.—Approximately 30.0 cm.
   Average leaf width.—Approximately 25.7 cm.
   Overall leaf blade shape.—Deltoid to narrow deltoid, pinnatisect.
   Aspect.—Leaf blade flat, but margins very wavy.
   Leaf apex.—Narrow acute.
   Leaf base.—Attenuate.
   Margin.—Pinnatisect, margins of lobes incised to pinnatifid.
   Texture of top surface.—Smooth, glossy.
   Texture of bottom surface.—Smooth, moderately glossy.
   Color.—Young foliage upper side: Green to yellow-green; in between near RHS 143B and 144A. Young foliage under side: Green to yellow-green; in between near RHS 143B and 144A. Mature foliage upper side: Green; near RHS N137C. Mature foliage under side: Green; near RHS N137B.
Leaflets: No leaflets present, leaves pinnatisect.
Venation:
   Type.—Pinnate.
   Venation coloration upper side.—Greyed-purple to brown; near RHS N186C and 200A.
   Venation coloration under side.—Brown; near RHS 200B.
Rachis description: No leaflets present, so no rachis present.
Other foliage characteristics: No
Other characteristics:
Spore production: No spores were produced up to date.
Disease resistance: Typical of Microsorum diversifolium.
Temperature tolerance: Typical of Microsorum diversifolium.
Drought tolerance: Typical of Microsorum diversifolium.

What is claimed is:

1. A new and distinct cultivar of Microsorum diversifolium plant named ‘Crested’ as herein illustrated and described.
